(19) United States
(12) Patent Application Publication
(10) Pub. No.: US 2016/0010120 A1
(43) Pub. Date: Jan. 14, 2016

(54) METHOD OF PREPARING BUTANOL FROM A BUTYRIC ACID-CONTAINING AQUEOUS FERMENTATIVE LIQUID

(71) Applicant: GREEN CELLULOSITY CORPORATION, HSINCHU CITY (TW)

(72) Inventors: CHIANG-HSIUNG TONG, HSINCHU CITY (TW); RUEY-FU SHIH, HSINCHU CITY (TW); YUN-HUIN LIN, HSINCHU CITY (TW); HOM-TI LEE, HSINCHU CITY (TW)

(21) Appl. No.: 14/794,279

(22) Filed: Jul. 8, 2015

Related U.S. Application Data
(60) Provisional application No. 62/021,880, filed on Jul. 8, 2014.

(30) Foreign Application Priority Data
Jun. 11, 2015 (TW) 104118890

(51) Int. Cl. C12P 7/16

(52) U.S. Cl. CPC C12P 7/16

(57) ABSTRACT

A method of preparing butanol from a butyric acid-containing aqueous fermentation broth is disclosed, wherein the butyric acid-containing aqueous fermentation broth is obtained from the fermentation of a substrate with the use of a microorganism.

This application claims the benefit of U.S. Provisional Patent Application No. 62/021,880, filed Jul. 8, 2014, the subject matters of which are incorporated herein by reference. Priority under 35 U.S.C. §119 is also claimed to TW Patent Application No. 104118890 filed on Jun. 11, 2015 , the entire content of which is incorporated herein by reference.

### FIELD OF THE INVENTION

The present invention relates to a method of manufacturing biobutanol; specifically, the present invention relates to a method of preparing butanol from a butyric acid-containing aqueous fermentation broth, wherein the butyric acid-containing aqueous fermentation broth is obtained from the fermentation of a substrate through the use of a microorganism.

### BACKGROUND OF THE INVENTION

Recently, with limited energy resources as well as decreasing petroleum resources, researches have been conducted about producing bioethanol as a fuel to help address the energy issue. The so-called “bioethanol” refers to ethanol produced from the fermentation of saccharides in crops by microorganisms, which may be used as a fuel alone or in combination with gasoline.

However, ethanol is not perfect as a fuel, wherein a known disadvantage is that: there is usually some degree of moisture in the environment, and when an ethanol-blended gasoline comes into contact with moisture in the environment, water will be absorbed into the ethanol-blended gasoline, such that ethanol and water mix together and then separate from gasoline, which is unfavorable to the subsequent use of the blended gasoline. In contrast, the butanol-blended gasoline does not absorb water even if water is introduced therein, and thus the separation of butanol and gasoline does not occur. Therefore, in the case of using butanol-blended gasoline, additional devices are not required in storage, transportation, and supply systems, and in vehicles capable of employing butanol-blended gasoline. Moreover, the vapor pressure of butanol is lower than that of ethanol, thereby decreasing the possibility of vapor lock in car engines. In addition, the air-fuel ratio of butanol is similar to that of the gasoline, which means that butanol may be mixed with gasoline in considerably greater amount without affecting the performance of engines.

Although there are many advantages for biobutanol as set forth above, butanol is more toxic to organisms compared with ethanol, and microbial strains producing butanol may lose the ability of producing butanol, thus making it difficult to accumulate enough butanol concentration in the fermentation broth. For example, in the case that a typical ABE (acetone-butanol-ethanol) fermentation is performed using Clostridium acetobutyricum, the ABE yield is as low as 0.2 g/h-L and the highest concentration of butanol in the fermentation broth is not more than about 1.3%, and thus a rather large fermentor is required. In addition, since the concentration of butanol in the fermentation broth is low, the energy required for separating and concentrating butanol from the fermentation broth is higher.

For the fermentation broth having a low concentration of butanol, it has been proposed to distill and obtain the biobutanol from the fermentation broth; however, the method requires an energy of 5,000 kcal or more for isolating 1 L of butanol, which is almost the heat of combustion of n-butanol (6,400 kcal/L) and is not economical. A liquid/liquid extraction method has been also proposed to recover butanol from the fermentation broth. For example, U.S. Pat. No. 4,260,836 by Sidney Levy discloses a liquid/liquid extraction method using a fermentation broth containing fluorocarbons with a high butanol extraction coefficient; U.S. Pat. No. 4,628,116 by Richard J. Cenedella discloses a method of liquid/liquid extraction for extracting butyric acid and butanol with vinyl bromide from a fermentation broth; and “In Situ Extractive Fermentation of Acetone and Butanol” (Biotech. and Bioeng., Vol. 31, P. 135-143, 1988) discloses a liquid/liquid extraction for extracting butanol with oleyl alcohol. However, since the butanol concentration in the fermentation broth is so low (not more than about 2%), the cost of isolation and purification required for the liquid/liquid extraction methods is still high. In addition, extractive solvents with high butanol extraction coefficient may deactivate the microbial strains used for producing butanol, thus making the liquid/liquid extraction less attractive.

U.S. Pat. No. 5,753,474 by David Edward Ramey proposes a two-step fermentation process comprising producing only butyric acid by Clostridium tyrobutylicum first, then selectively producing only butanol by Clostridium acetobutyricum. The use of a fermentor which immobilizes microorganisms on a fibrous bed increases the yield to 6 g/h-L, but the highest concentration of butanol in the fermentation broth is still not more than about 2%.

In addition, a method has been proposed that comprises extracting butyric acid from a fermentation broth first, then converting the butyric acid to butanol. “Extractive Fermentation for Butyric Acid Production from Glucose by Clostridium tyrobutylicum” (Biotechnol Bioeng. 2003 Apr. 5; 82(1):93-102), discloses transferring a fermentation broth from a fibrous bed reactor to a hollow fibrous film extraction column, and performing a reactive extraction by using water-insoluble trialkylamine (such as Alamine 336) as an extracting agent in the extraction column to obtain trialkylammonium butyrate. Then, the trialkylammonium butyrate is transferred to another hollow fibrous film extraction column using sodium hydroxide as an extracting agent to regenerate the trialkylamine, to obtain an aqueous solution of sodium butyrate in high concentration. Hydrochloric acid is then added to the aqueous solution of sodium butyrate to obtain an aqueous solution of butyric acid. Although the method produces butyric acid in high purity, a large amount of acid and base is required (1 mole of sodium hydroxide and 1 mole of hydrochloric acid are required to prepare 1 mole of butyric acid) and thus is not desirable.

Therefore, an economically practical method of manufacturing biobutanol which is able to prepare butanol from fermentation broth in high yield is needed. The present invention thus provides a method of preparing biobutanol from a butyric acid-containing aqueous fermentation broth in high yield, wherein most of the materials added in each step in the method of the present invention can be separated in the subsequent operation and further recycled and used, thereby improving the economic efficiency.

### SUMMARY

At least an embodiment provides a method of preparing butanol from a butyric acid-containing aqueous fermentation broth, wherein the butyric acid-containing aqueous fermentation broth is obtained from the fermentation of a substrate with the use of a microorganism, the method comprising the following steps:
a) mixing the butyric acid-containing aqueous fermentation broth with a trialkylamine-containing extractive solvent to perform a liquid/liquid extraction and obtain an aqueous phase and a non-aqueous phase, wherein the trialkylamine is water-insoluble and the non-aqueous phase contains a trialkylammonium butyrate;
b) heating the non-aqueous phase obtained from step a) to decompose the trialkylammonium butyrate in the non-aqueous phase to obtain butyric acid and trialkylamine;
c) esterifying the butyric acid obtained from step b) with methanol to obtain methyl butyrate; and
d) hydrogenolyzing the methyl butyrate obtained from step c) to obtain butanol and methanol.

### BRIEF DESCRIPTION OF THE DRAWINGS

FIG. 1 is a diagram of an exemplary reaction process wherein a carbohydrate aqueous solution 1 as a raw material sequentially passes through a fermentor 100, a pH adjusting tank 200, an extractor 300, a heater 400, an esterification reactor 500, a hydrogenolysis reactor 600, and a distillation column 700 to provide butanol 13.

FIG. 2 is a diagram of an extraction process according to an example.

### DESCRIPTION OF THE PREFERRED EMBODIMENTS

Hereinafter, some embodiments in accordance with the present invention will be described in detail. However, the present invention may be applied to various embodiments without departing from the spirit of the present invention. As such, the scope of the present invention shall not be considered to be limited to what is illustrated herein. Furthermore, unless it is additionally indicated, the expressions “a”, “the”, and the like recited in the specification of the present invention (especially in the claims) include both the singular and plural forms of the noun that follows.

At least an embodiment of the present invention provides a method of preparing butanol from a butyric acid-containing aqueous fermentation broth, wherein the butyric acid-containing aqueous fermentation broth is obtained from the fermentation of a substrate with the use of a microorganism, the method comprising the following steps:
a) mixing the butyric acid-containing aqueous fermentation broth with a trialkylamine-containing extractive solvent to perform a liquid/liquid extraction and obtain an aqueous phase and a non-aqueous phase, wherein the trialkylamine is water-insoluble and the non-aqueous phase contains a trialkylammonium butyrate;
b) heating the non-aqueous phase obtained from step a) to decompose the trialkylammonium butyrate in the non-aqueous phase to obtain butyric acid and trialkylamine;
c) esterifying the butyric acid obtained from step b) with methanol to obtain methyl butyrate; and
d) hydrogenolyzing the methyl butyrate obtained from step c) to obtain butanol and methanol.

In the method, a butyric acid-containing aqueous fermentation broth is used to prepare butanol, wherein the butyric acid-containing aqueous fermentation broth may be any one that is obtained from the fermentation of a substrate with the use of a microorganism. Preferably, the substrate includes carbohydrate. A carbohydrate aqueous solution may be added to a fermentor filled with a carrier to perform fermentation to provide the butyric acid-containing aqueous fermentation broth, wherein a microorganism that is able to convert the carbohydrate into butyric acid by fermentation is fixed on the carrier. The carbohydrate is selected in accordance with the microorganism used. Preferably, a mixed culture system comprising strains having the ability to produce butyric acid and strains having the ability of carbon fixation is used in the fermentor to convert the carbohydrate into butyric acid in a low carbon-loss way. The so called “ability of carbon fixation” means the ability of using CO and/or CO2 as a carbon source and converting them to organic matters.

Examples of strains used for producing butyric acid comprise, but are not limited to, Clostridium tyrobutylicum, Clostridium butylicum, Clostridium acetobutyricum, Clostridium beuerinckii, and combinations thereof. Examples of strains having the ability of carbon fixation comprise, but are not limited to, Clostridium Coskatii, Clostridium Ljungdahlii, Clostridium difficile, Clostridium authoethanogenium, Clostridium ragsdalei, or combinations thereof.

The substrate may be any substances, which can be metabolized by microorganisms as sources to conduct fermentation, including carbohydrate, organic acid, gaseous substrate (CO and/or CO₂), or a combination thereof. Preferably, the substrate includes carbohydrates. The carbohydrates may be such as saccharides, comprising monosaccharides, disaccharides, polysaccharides, and combinations thereof. The monosaccharides may be such as pentose, hexose, glucose, xylose, galactose, or any combinations thereof; the disaccharides may be such as lactose, sucrose, cellobiose, or any combinations thereof; the polysaccharides may be such as starch, glycogen, cellulose, or any combinations thereof. Examples of the carbohydrate aqueous solution comprise such as an aqueous solution of glucose, a juice of sugar cane, a corn steep liquor, or a pentose-hexose mixture obtained from the hydrolysis of a lignin material. The carbohydrate aqueous solution may also be a solution containing soluble saccharides obtained from processing biomasses through a saccharification process. Suitable biomasses comprise, but are not limited to, such as corn stover, corncob, straw, maize fiber and the like.

In addition to saccharides, the carbohydrate aqueous solution may also comprise an organic acid, such as lactic acid or acetic acid. In an embodiment of the present invention, the carbohydrate aqueous solution may comprise lactic acid and at least one saccharide, wherein the weight ratio of the lactic acid to the at least one saccharide is 0.1:1 to 10:1, such as 0.3:1 to 3:1, preferably 0.5:1 to 1.5:1. In an example, the weight of the lactic acid is more than or equal to the weight of the at least one saccharide. It is discovered that the fermentation using a carbohydrate aqueous solution simultaneously comprising an organic acid (such as lactic acid) and saccharides produced less carbon dioxide, compared to a carbohydrate aqueous solution comprising saccharides alone, thereby decreasing the loss of carbon yield and increasing the carbon yield of butyric acid. As illustrated in the following examples, in an embodiment of the present invention, a solution comprising glucose and lactic acid is used as the carbohydrate aqueous solution, and Clostridium tyrobutylicum is used as the microorganism to perform fermentation and provide the butyric acid-containing aqueous fermentation broth.

According to the method, a butyric acid-containing aqueous fermentation broth is mixed with a trialkylamine-containing extractive solvent to perform a liquid/liquid extraction and obtain an aqueous phase and a non-aqueous phase in step a), wherein the trialkylamine is water-insoluble and the non-aqueous phase contains a trialkylammonium butyrate. Step a) may be performed at any temperature as long as the two-phase state, i.e. the non-aqueous phase and the aqueous phase, is maintained.

The extractive solvent used in step a) may be the water-insoluble trialkylamine itself or a mixture comprising the water-insoluble trialkylamine. Preferably, the water-insoluble trialkylamine has the formula of NR₁R₂R₃, in which R₁, R₂, and R₃ are the same or different and are each independently C5-C10 alky. More preferably, the water-insoluble trialkylamine used in step a) has the formula of NR₁R₂R₃, wherein R₁, R₂, and R₃ are the same alkyl; for example, the water-insoluble trialkylamine may be selected from the group consisting of tripentylamine, trihexylamine, trioctylamine, tridecylamine, and combinations thereof.

Optionally, the extractive solvent in step a) may comprise a diluent to increase the fluidity of the extractive solvent, thus increasing the extraction efficiency. Any suitable diluent may be used as long as the diluent is miscible with the trialkylamine and does not vigorously react with the trialkylamine or butyric acid in the following heating step. Examples of suitable diluent comprise, but are not limited to, such as acetophenone, kerosene, n-butanol and the like.

According to the method, the butyric acid in the fermentation broth reacts with the trialkylamine by the liquid/liquid extraction in step a) which produces water-insoluble trialkylammonium butyrate so as to obtain the non-aqueous phase containing the trialkylammonium butyrate. Then, the non-aqueous phase is heated in step b) to decompose the trialkylammonium butyrate therein to obtain butyric acid and trialkylamine.

Step b) may be performed by any common heating methods in the art. For example, step b) may be performed by evaporation using an evaporator, or by distillation using a distillation column, but is not limited thereto. The suitable temperature in step b) is selected depending on the water-insoluble trialkylamine used in step a). For example, when tripentylamine is used as the water-insoluble trialkylamine in the extractive solvent so as to obtain tripentylammonium butyrate, since tripentylammonium butyrate decomposes at a temperature of 90 to 100° C., it is possible to perform step b) at the temperature of 90 to 100° C. or more to obtain butyric acid and tripentylamine. When step b) is performed by distillation, butyric acid may be isolated from the top of the column, and tripentylamine may be isolated from the bottom of the column, such that butyric acid may enter step c) and tripentylamine may be recycled to be used in step a).

According to the method of the present invention, the butyric acid obtained in step b) is esterified with methanol to obtain methyl butyrate in step c). The esterification may be performed in the presence of an esterification catalyst, under the conditions of such as a temperature of 80 to 300° C., a pressure of 1 to 20 atm, and an amount of 1 to 10 moles methanol per mole of butyric acid. Generally, when the reaction temperature is lower than 80° C., the catalytic activity is low and thus the conversion decreases; on the contrary, when the reaction temperature is higher than 300° C., more by-products may be formed and thus the selectivity decreases.

Step c) is preferably performed at lower pressures, which brings the following advantages: the lower pressures are beneficial to the evaporation of some feed, thereby performing the esterification at a gas-liquid coexistent state, and increasing the thermodynamic equilibrium conversion. In addition, it is discovered that the conversion will be lower to 70 to 80% when the amount ratio of methanol and butyric acid is close to the stoichiometric ratio (1:1). Therefore, it is preferable to use excessive amount of methanol in step c) to increase the conversion of butyric acid. In general, in the case of using 2 or more moles of methanol per mole of butyric acid, the conversion may be 95% or more; however, if the molar ratio of methanol to butyric acid is too high, more by-products may be formed, resulting in lowering the selectivity.

Homogeneous or heterogeneous esterification catalysts may be used in step c). Examples of homogeneous esterification catalysts comprise, but are not limited to, such as sulfuric acid, hydrochloric acid, or nitric acid. Examples of heterogeneous esterification catalysts comprise, but are not limited, to such as ion exchange resin, zeolite, siallite, alumina, sulfonated carbon, and heteropoly acid.

In an embodiment according to the present invention, the esterification of step c) is performed in the presence of an esterification catalyst, under the conditions of temperature of 60 to 140° C., pressure of 1 to 4 atm, and an amount of 1 to 7 moles methanol per mole of butyric acid, wherein the catalyst may be selected from the group consisting of ion exchange resin, zeolite, siallite, alumina, sulfonated carbon, heteropoly acid, and combinations thereof

According to the method of the present invention, the methyl butyrate obtained in step c) is hydrogenolyzed in step d) to obtain butanol and methanol. The hydrogenolysis may be performed in the presence of a hydrogenolysis catalyst. Generally, along with the increase of the reaction temperature, the conversion of the hydrogenolysis increases and the selectivity of the product decreases; while along with the increase of the reaction pressure, the conversion of the hydrogenolysis increases. Therefore, the reaction conditions are generally adjusted depending on the properties of the hydrogenolysis catalyst used and is not limited to the disclosure herein. In an embodiment according to the present invention, the hydrogenolysis of step d) is performed in the presence of a hydrogenolysis catalyst, under the conditions of temperature of 120 to 300° C., pressure of 1 to 100 atm, and an amount of 1 to 100 moles of hydrogen per mole of methyl butyrate.

Any suitable hydrogenolysis catalyst may be used to perform step d) of the present invention. For example, but not limited thereof, the hydrogenolysis catalyst may be one or more selected from the group consisting of copper, zinc, chromium, nickel, cobalt, silver, molybdenum, palladium, ruthenium, rhodium, and oxides thereof. The hydrogenolysis catalyst may also be used as supported on a suitable support. For example, in an embodiment of the present invention, catalytic copper supported on silicon dioxide, i.e. Cu/SiO_{2,} is used as the hydrogenolysis catalyst.

By means of the hydrogenolysis of step d), methyl butyrate is decomposed to obtain butanol and methanol, wherein the butanol may be used as a fuel and the methanol may be recycled to be used in step c).

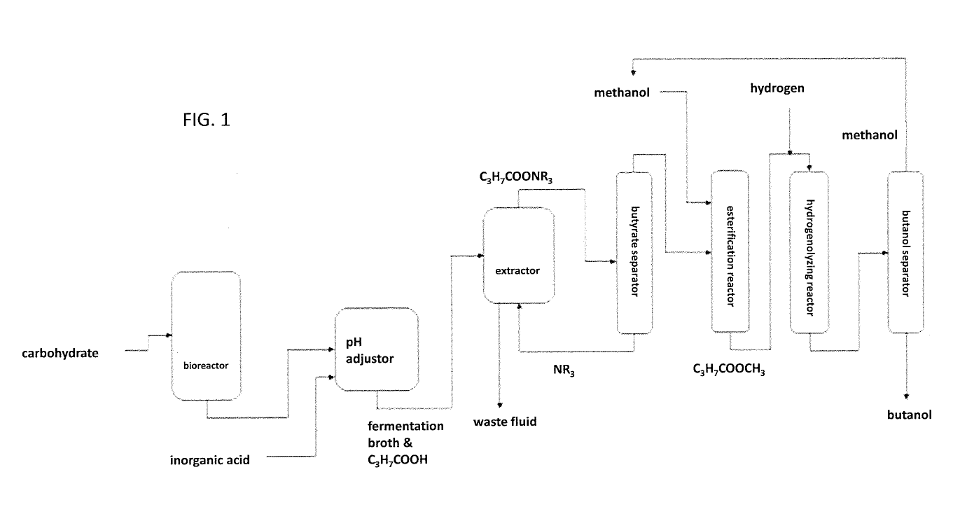

An embodiment of the present invention is hereby described in accordance with FIG. 1. As illustrated in FIG. 1, the carbohydrate aqueous solution 1 is fed into a fermentor 100 to perform fermentation, so as to produce an aqueous fermentation broth 2. Then, prior to performing extraction, the pH value of the aqueous fermentation broth 2 may be optionally adjusted by a pH adjusting tank 200 to a suitable range to obtain an aqueous fermentation broth 4. Then, the aqueous fermentation broth 4 is fed to an extractor 300 and is extracted by an extractive solvent 5 which comprises water-insoluble trialkylamine (such as tripentylamine) to obtaining a non-aqueous phase extract 6 containing trialkylammonium butyrate and a raffinate 7. The non-aqueous phase extract 6 is fed to a heater 400 and decomposed to obtain butyric acid 8 and trialkylamine 5, in which the trialkylamine 5 is recycled back to the extractor 300. Then, the butyric acid 8 is fed to an esterification reactor 500 to perform esterification with methanol 9, to obtain methyl butyrate 10. The methyl butyrate 10 is then fed to a hydrogenolysis reactor 600 provided with hydrogen 11 to perform hydrogenolysis, so as to obtain a liquid mixture of butanol and methanol 12. The liquid mixture of butanol and methanol 12 is then provided to a distillation column 700, separated into the product butanol 13 and methanol 9, in which the methanol 9 is recycled to the esterification reactor 500.

As described above, the method of the present invention starts with mixing a water-insoluble trialkylamine-containing extractive solvent with a butyric acid-containing aqueous fermentation broth to perform a liquid/liquid extraction, heating the obtained non-aqueous phase to decompose the trialkylammonium butyrate therein to obtain butyric acid and trialkylamine, then esterifying the obtained butyric acid with methanol, and hydrogenolyzing the methyl butyrate obtained therefrom, thereby preparing butanol from the fermentation broth. Since the butyric acid comprised in the fermentation broth can be extracted with a water-insoluble trialkylamine-containing extractive solvent in high yield, and the subsequent esterification of butyric acid with methanol and the hydrogenolysis of methyl butyrate can also by performed in high yield, the object of preparing butanol in high yield can be achieved in the present invention. In addition, for the materials used in the method of the present invention including the water-insoluble trialkylamine and the methanol, may be isolated in the following operation and further recycled and used, thereby improving the economic efficiency. Furthermore, the butyric acid and the methyl butyrate obtained in the method of the present invention may be optionally used as products, thereby improving the applicability.

### EXAMPLES

Embodiments of the present invention are further illustrated by the following examples, wherein the analysis methods employed are as follows.

Optical density: The optical density of the cell suspension was measured by a spectrophotometer (OPTIZEN, model 2120UV plus) at a wavelength of 600 nm (OD600) to analyze the free cell density.

Concentrations of saccharides and organic acids in the reaction solution: Solutions comprising organic acids and saccharides are analyzed by a high performance liquid chromatograph (HPLC) (Agilent HP-1100) disposed with an Aminex HPX-87H column (300×7.8 mm), a 75° C. column oven, and a refractive index detector. The mobile phase was 18 mM sulfuric acid with a flow rate of 6 mL/minute. The concentrations of saccharides and organic acids were determined according to the standard calibration curve.

Composition of gas: Gas was analyzed by a gas chromatograph (GC) (YL 6100 GC) disposed with a ShinCarbon ST 100/120 mesh column (2 m×1 mm ID micropacked). The temperatures of the injector and the detector were respectively set to 100° C. and 200° C. The carrier gas was helium with a flow rate of 10 mL/minute. The carbon dioxide peak was defined by comparing the retention time with the standard retention time.

### Preparation Example

### Preparation of the Butyric Acid-Containing Aqueous Fermentation Broth

[Microorganism Culture]

Clostridium tyrobutylicum (ATCC 25755) used as the microorganism in the preparation example was purchased from Bioresource Collection and Research Center (BCRC). A stock culture was anaerobically pre-cultured in a serum bottle containing 100 mL Reinforced Clostridial Medium (RCM) (purchased from Merck), with stirring at 37° C. for 48 hours before use.

A basal medium was prepared, which comprises the following components per liter of deionized water: 5 g yeast extract, 5 g peptone, 3 g ammonium sulfate, 1.5 g potassium dihydrogen phosphate, 0.6 g MgSO₄.7H₂O, and 0.03 g FeSO₄.7H₂O (see Wu et al., Biotechnology and Bioengineering 2003, 82(1), 93-102). Then, glucose and/or lactic acid as a carbon source were added to the basal medium to prepare a feedstock. When the feedstock was used in microorganism mass culturing to perform cell immobilization, the concentration of glucose in the prepared feedstock was 10 g/L. When the feedstock was used in a batch fermentation to produce organic compounds, the concentration of glucose in the prepared feedstock was 15 g/L and the concentration of lactic acid was 15 g/L. The feedstock was sterilized by autoclave at 121° C. and 15 psig for 30 minutes before use.

[Cell Immobilization]

300 mL of the microorganism suspension prepared in the serum bottle was in a 5 L fermentation tank filled with 4 L feedstock, the feedstock comprising 10 g/L glucose as the carbon source. The microorganism was then allowed to grow for 7 days until the concentration of the microorganism cells reached approximately an optical density of 5 (OD 600).

The cells were immobilized with phosphorylated polyvinyl alcohol (PVA) gel granules according to the method disclosed by Chen, K. (see “Immobilization of microorganism with phosphorylated polyvinyl alcohol (PVA) gel,” Enzyme Microbiob. Technol. 1994, 16, 79-83, which is incorporated by reference herein in its entirety). The microorganism suspension was centrifuged in a centrifuge (KUBOTA, 7780) for 10 minutes at 6,500 rpm to collect the cells. The collected cells were added to a PVA aqueous solution to obtain a suspension mixture of about 9% w/v suspension content, i.e. 20 g wet cells per liter of PVA solution. The PVA-cell mixed solution was then dropwise added into a solution of concentrated boric acid and sodium phosphate and mildly stirred for 1 to 2 hours to form granules with immobilized microorganism cells, having a diameter of 3 to 4 mm. Then, the granules were washed with water.

[Batch Fermentation]

Batch fermentation was performed in a 2 L agitator fermentation tank. The fermentation tank was filled with feedstock solution comprising 15 g/L glucose and 15 g/L L-lactic acid as the carbon source, aerated with nitrogen to an anaerobic condition, and the pH value was adjusted to 6.0 with 2N sodium hydroxide. Then, 70 g of the PVA granules with immobilized Clostridium tyrobutylicum cells, as prepared in the previous step, was inoculated, the inoculum size being about 5% w/v. The fermentation was performed at 37° C. and stirring at 600 rpm, adding 2N sodium hydroxide solution from time to time to maintain the pH value at about 6±0.1. The fermentation was kept until butyric acid was no longer produced due to product inhibition. The compositions of liquid and gas in the fermentation tank were analyzed, and it was determined that the final butyric acid concentration was 17.2 g/L, the carbon yield of butyric acid was 83%, and carbon loss due to production of carbon dioxide did not occur.

### Example 1

### Obtaining Butyric Acid by Extraction from Fermentation Broth

[Extraction with Trioctylamine]

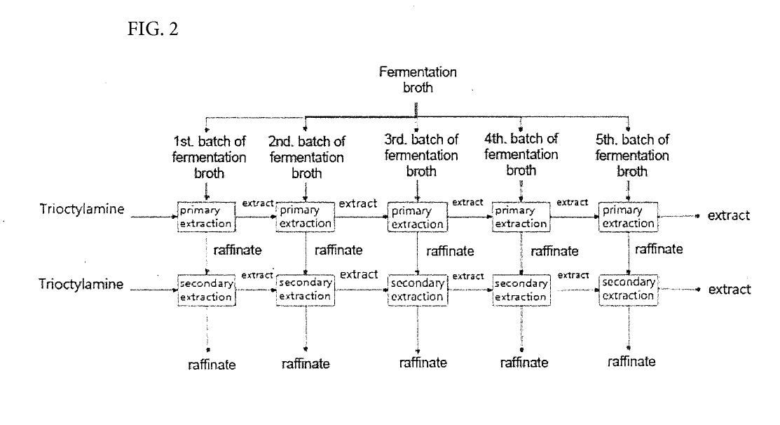

18,941 g of fermentation broth was provided, which had a pH value of 6.6 and comprised 2.4 weight % of butyric acid. The pH value of the fermentation broth was first adjusted to about 2.43 with sulfuric acid. Then, as shown in FIG. 2, the fermentation broth was divided into five batches, each having the weight of 4,000, 3,675, 4,000, 3,500, and 3,766 g. Each batch of the fermentation broth was extracted with trioctylamine by two-stage cross current extraction. At the primary extraction, 500 g trioctylamine was mixed with the fermentation broth to perform extraction, while the pH value of the aqueous phase was maintained at about 1.28 with sulfuric acid. At the secondary extraction, 487 g trioctylamine was mixed with the primary raffinate (i.e. the aqueous phase) to perform extraction, while the pH value of the aqueous phase was maintained at about 2.58 with sulfuric acid. The extracts (i.e. the non-aqueous phase) from each stage were mixed with the next batch of the fermentation broth to perform two-stage cross current extraction as well. After the five batches of fermentation broth were sequentially extracted, 719 g of primary extract and 678 g of secondary extract comprising trioctylammonium butyrate were obtained.

The extraction efficiency of the above extraction process was determined by the following method (see Shu et al. “Conditions of separating butyric acid from fermentation broth by complexation extraction” CHEMICAL ENGINEERING (CHINA) 2011, Vol. 39(8), which is incorporated by reference herein in its entirety). Sodium hydroxide was added to both the obtained primary and secondary extracts, followed by reaction to form sodium butyrate. The concentrations of sodium butyrate obtained from the obtained primary and secondary extracts were then determined with high performance liquid chromatograph (HPLC), both being 19 weight %.

[Heating the Extract to Obtain Butyric Acid]

1,397 g of the extract in the previous step was placed in a flask and heated to boil at a pressure of 30 mmHg, and the resulting evaporated vapor was condensed by a condenser on top of the flask, to obtain a liquid (313 g) with 76.8 weight % of butyric acid. Then the liquid was distilled in a batch distillation column at a pressure of 30 mmHg, thereby collecting a distillate liquid (133 g) comprising 97.5 weight % of butyric acid and 2.4 weight % of propionic acid.

The above results showed that by mixing the water-insoluble trialkylamine-containing extractive solvent with the butyric acid-containing aqueous fermentation broth to perform a liquid/liquid extraction in step a) of the present invention, butyric acid can be easily isolated from the fermentation broth in the form of trialkylammonium butyrate in high yield. In addition, in the heating procedure of step b) the trialkylammonium butyrate in the non-aqueous phase (i.e. the extract liquid) from step a) decomposes to obtain butyric acid solution in high purity.

### Examples 2 to 4

### Esterfication

### Example 2

Butyric acid of high purity (Alfa Aesar, 99+%, CAS No.: 107-92-6), methanol, and Amberlyst 35 wet ion-exchange resin (purchased from Rohm & Haas) as the acidic catalyst were added to a 250 mL three-necked flask having a reflux condenser, with stirring, wherein the molar ratio of butyric acid to methanol was 1:2, and the concentration of the acidic catalyst was 20 g/L. The reactants were heated to 80° C. and maintained at 80° C. to perform esterification for 2 hours. The molar yield of obtained methyl butyrate was 64.9%.

### Example 3

The procedures of Example 2 were repeated, except for the molar ratio of butyric acid to methanol being 1:3, the concentration of the acidic catalyst being 100 g/L, and the reaction time being 75 minutes. The molar yield of obtained methyl butyrate was 89.8%.

### Example 4

The procedures of Example 2 were repeated, except for the molar ratio of butyric acid to methanol being 1:4, the concentration of the acidic catalyst being 200 g/L, and the reaction time being 75 minutes. The molar yield of obtained methyl butyrate was 99.9%.

As shown in Examples 2 to 4, the butyric acid obtained from step b) was esterified with methanol to obtain methyl butyrate in step c) of the method of the present invention, wherein the yield can reach 99.9% by adjusting the reaction conditions and the molar ratio of butyric acid to methanol. In addition, since methanol is used in the esterification in the method of the present invention, in the case of using excess methanol to increase the conversion, the residual methanol (boiling point 64.7° C.) can be easily isolated and recycled from the product of methyl butyrate (boiling point 102° C.) due to the distinct difference in boiling points.

### Example 5

### Hydrogenolysis-I

Copper-chromium catalyst (from Strem company) is loaded into a packed bed reactor (length 55 cm, inner diameter 1.32 cm, outer diameter 1.8 cm). Hydrogenolysis of methyl butyrate was performed by providing hydrogen at a rate of 1100 mL/minute under each of the sets of operating conditions as listed in Table 1. The results are shown in Table 1.

TABLE 1

| | | | Flow rate of | | |
|---|---|---|---|---|---|
| | | | methyl | Conversion of | Yield of |
| Catalyst | Temperature | Pressure | butyrate | methyl butyrate | butanol |
| (g) | (° C.) | (psi) | (g/hr) | (%) | (%) |
| 38 | 210 | 1000 | 6 | 63.04 | 44.85 |
| 38 | 220 | 1000 | 6 | 99.61 | 87.38 |
| 60 | 180 | 800 | 2 | 88.797 | 60.160 |
| 60 | 200 | 800 | 2 | 98.576 | 80.873 |
| 60 | 210 | 800 | 2 | 99.623 | 78.481 |
| 60 | 200 | 300 | 6 | — | 95.2 |
| 60 | 200 | 400 | 6 | — | 96.3 |
| 60 | 200 | 500 | 6 | — | 97.3 |
| 60 | 200 | 600 | 6 | — | 99.5 |

### Example 6

### Hydrogenolysis-II

[Preparation of Cu/SiO₂ Catalyst]

30 g of CuNO₃.3H₂O and 200 mL deionized water were added into a beaker and stirred, then the mixture solution was titrated with sodium hydroxide aqueous solution to a pH value of 8 to 9, forming a slurry solution. After 44 mL of tetraethyl silicate (comprising 28% SiO₂) was added to the slurry solution and mixed by stirring, the solution was put into a batch reactor and heated to 80° C. to react and age for 4 hours. Then, the product was suction filtrated and put into an oven to dry at 120° C. for 16 hours, obtaining a Cu/SiO₂ catalyst. After the obtained catalyst product was compressed into tablets and dried, the tablets were air calcinated at 400° C. for 4 hours, and reduced with hydrogen at a flow rate of 1500 mL/minute at 240° C. for 4 hours.

[Hydrogenolysis]

Devices identical to those used in Example 5 were used, but with the reaction tube filled with 20 g of catalyst prepared in the present Example. Hydrogenolysis of methyl butyrate was performed under the flow rates of methyl butyrate and of hydrogen as listed in Table 2, with the results being shown in Table 2.

TABLE 2

| | | | Flow | | |
|---|---|---|---|---|---|
| | | | rate of | | |
| | | Flow rate of | methyl | Conversion of | Yield of |
| Temperature | Pressure | hydrogen | butyrate | methyl butyrate | butanol |
| (° C.) | (psi) | (L/min) | (g/hr) | (%) | (%) |
| 240 | 1000 | 1.5 | 6 | 96.54 | 93.21 |
| 240 | 1000 | 1.5 | 4 | 98.92 | 97.48 |
| 240 | 1000 | 0.75 | 6 | 92.5 | 88.5 |

As shown in Examples 5 and 6, the methyl butyrate obtained from step c) was hydrogenolyzed to obtain butanol and methanol in step d) of the method of the present invention, which was performed under the appropriate reaction conditions, rather than extreme conditions, and the conversion of methyl butyrate and the yield of butanol were both near 100% with nearly no reaction loss.

### Comparative Example 1

The procedures of Example 5 were repeated, except for ethyl butyrate being used as the reactant, the flow rate of hydrogen being 1100 mL/hour, the amount of catalyst being 60 g, and the reaction conditions being those listed in Table 3. The results are also shown in Table 3.

TABLE 3

| | | Flow rate of | | | |
|---|---|---|---|---|---|
| | | ethyl | Conversion of | Yield of | Yield of |
| Temperature | Pressure | butyrate | ethyl butyrate | ethanol | butanol |
| (° C.) | (psi) | (g/hr) | (%) | (%) | (%) |
| 180 | 1000 | 2 | 91.864 | 83.413 | 79.570 |
| 190 | 1000 | 2 | 99.293 | 90.156 | 85.180 |
| 200 | 1000 | 2 | 97.420 | 11.538 | 96.634 |

### Comparative Example 2

The procedures of Example 5 were repeated, except for butyl butyrate being used as the reactant, the flow rate of hydrogen being 1100 mL/hour, and the reaction conditions being those listed in Table 4. The results are also shown in Table 4.

TABLE 4

| | | | Flow rate of | | |
|---|---|---|---|---|---|
| | | | butyl | Conversion of | Yield of |
| Catalyst | Temperature | Pressure | butyrate | butyl butyrate | butanol |
| (g) | (° C.) | (psi) | (g/hr) | (%) | (%) |
| 20 | 230 | 1000 | 4 | 40.3 | 40.2 |
| 40 | 230 | 1000 | 4 | 95.3 | 61.4 |

### Comparative Example 3

The procedures of Example 5 were repeated, except for each using methyl butyrate, ethyl butyrate, or butyl butyrate as the reactant, the flow rate of hydrogen being 1100 mL/hour, and the weight hourly space velocity (WHSV) being 0.1/hour. For different reactants the obtained highest yields are listed in Table 5 for comparison.

TABLE 5

| | | | | Flow rate of | Yield of |
|---|---|---|---|---|---|
| | Catalyst | Temperature | Pressure | butyrate | butanol |
| Reactant | (g) | (° C.) | (psi) | (g/hr) | (%) |
| methyl butyrate | 60 | 200 | 600 | 6 | 99.5 |
| ethyl butyrate | 60 | 220 | 300 | 6 | 96.5 |
| ethyl butyrate | 60 | 200 | 400 | 6 | 82.3 |
| butyl butyrate | 60 | 200 | 500 | 6 | 95.5 |

As shown in the above Example 5 and Comparative Examples 1 to 3, a higher yield of butanol was achieved by the method of the present invention using methyl butyrate as the hydrogenolysis reactant, compared with the highest yields using ethyl butyrate or butyl butyrate as the reactant. In addition, by comparing the results in Comparative Example 3 and the results in Example 5 (Table 1), it was discovered that the method of the present invention using methyl butyrate as the reactant indeed provides a higher yield of butanol under the same reaction condition (60 g of catalyst, a reaction temperature of 200° C., a reaction pressure of 400 or 500 psi, and a flow rate of butyrate 6 g/hr). Furthermore, the products of butanol and methanol obtained from the hydrogenolysis of methyl butyrate, according to the present invention, can be easily separated due to the distinct difference in boiling points.

The above disclosure is related to the detailed technical contents and inventive features thereof. People skilled in this field may proceed with a variety of modifications and replacements based on the disclosures and suggestions of the invention as described without departing from the characteristics and spirit thereof. Nevertheless, although such modifications and replacements are not fully disclosed in the above descriptions, they have substantially been covered in the following claims as appended.

What is claimed is:

1. A method of preparing butanol from a butyric acid-containing aqueous fermentation broth, wherein the butyric acid-containing aqueous fermentation broth is obtained from the fermentation of a substrate with the use of a microorganism, the method comprising the following steps:
a) mixing the butyric acid-containing aqueous fermentation broth with a trialkylamine-containing extractive solvent to perform a liquid/liquid extraction and obtain an aqueous phase and a non-aqueous phase, wherein the trialkylamine is water-insoluble and the non-aqueous phase contains a trialkylammonium butyrate;
b) heating the non-aqueous phase obtained from step a) to decompose the trialkylammonium butyrate in the non-aqueous phase to obtain butyric acid and trialkylamine;
c) esterifying the butyric acid obtained from step b) with methanol to obtain methyl butyrate; and
d) hydrogenolyzing the methyl butyrate obtained from step c) to obtain butanol and methanol.

2. The method according to claim 1, wherein the microorganism is selected from the group consisting of Clostridium tyrobutylicum, Clostridium butylicum, Clostridium acetobutyricum, Clostridium beijerinckii, and combinations thereof.

3. The method according to claim 1, wherein in step a), the water-insoluble trialkylamine has the formula of NR₁R₂R₃, in which R₁, R₂, and R₃ are the same or different and are each independently C5-C10 alkyl.

4. The method according to claim 3, wherein R₁, R₂, and R₃ are the same alkyl.

5. The method according to claim 3, wherein the water-insoluble trialkylamine is selected from the group consisting of tripentylamine, trihexylamine, trioctylamine, tridecylamine, and combinations thereof.

6. The method according to claim 1, wherein the step c) is performed in the presence of an esterification catalyst, under the conditions of a temperature of 60 to 140° C., a pressure of 1 to 4 atm, and an amount of 1 to 7 moles methanol per mole of butyric acid.

7. The method according to claim 6, wherein the esterification catalyst is a solid acid catalyst with superacidity selected from the group consisting of ion exchange resin, zeolite, siallite, alumina, sulfonated carbon, heteropoly acid, and combinations thereof.

8. The method according to claim 1, where the step d) is performed in the presence of a hydrogenolysis catalyst, under the conditions of a temperature of 120 to 300° C., a pressure of 1 to 100 atm, and an amount of 1 to 100 moles hydrogen per mole of methyl butyrate.

9. The method according to claim 8, wherein the hydrogenolysis catalyst is selected from the group consisting of copper, zinc, chromium, nickel, cobalt, silver, molybdenum, palladium, ruthenium, rhodium, oxides thereof, and combinations thereof.

10. The method according to claim 9, wherein the hydrogenolysis catalyst is supported on a support.

11. The method according to claim 10, wherein the hydrogenolysis catalyst is Cu/SiO₂.

12. The method according to claim 1, further comprising feeding the trialkylamine obtained in step b) to step a).

13. The method according to claim 1, further comprising feeding the methanol obtained in step d) to step c).

14. The method according to claim 12, further comprising feeding the methanol obtained in step d) to step c).
